(19) United States
(12) Patent Application Publication
(10) Pub. No.: US 2024/0197804 A1
(43) Pub. Date: Jun. 20, 2024

(54) PHARMACEUTICAL AND NUTRACEUTICAL COMPOSITION COMPRISING A MIXTURE OF PLANTS FOR THE PREVENTION AND TREATMENT OF NEURODEGENERATIVE DISEASES

(71) Applicant: LABORATOIRE AROMA SCIENCE, SAUCATS (FR)

(72) Inventor: Laurent MOY, SAUCATS (FR)

(21) Appl. No.: 18/287,342

(22) PCT Filed: Apr. 22, 2022
(86) PCT No.: PCT/EP2022/060761

§ 371 Date: Nov. 8, 2023

(30) Foreign Application Priority Data
Apr. 23, 2021 (FR) FR2104264

(51) Int. Cl. A61K 36/61; A61K 36/28; A61K 36/324; A61K 36/54; A61K 36/835; A61K 36/8905; A61P 25/28

(52) U.S. Cl. CPC A61K 36/61; A61K 36/28; A61K 36/324; A61K 36/54; A61K 36/835; A61K 36/8905; A61P 25/28

(57) ABSTRACT

The present invention relates to the pharmaceutical and nutraceutical fields and, more specifically, to a composition comprising an extract from a combination of plants for the prevention and/or treatment of a neurodegenerative disease

### FIELD OF THE INVENTION

The present invention relates to the pharmaceutical and nutraceutical fields and, more specifically, to a composition comprising as active ingredient at least one extract derived from a combination of plants.

The composition according to the invention is used for the prevention and/or treatment of neurodegenerative diseases, in particular Alzheimer's disease.

### PRIOR ART

Alzheimer's disease (AD) is a neurodegenerative disease of the brain tissue which mainly affects people over 65 years of age. Besides age, other risk factors have been identified, such as genetic factors or environmental factors. This disease is characterized by a degenerative disorder of the central nervous system associated with a significant loss of specific neural cells. It is clinically manifested by a progressive loss of the memory, cognition, reasoning, judgment, and emotional stability of the patient, which gradually leads to deep mental deterioration and finally to their death.

Therefore, Alzheimer's disease constitutes a major public health issue given the progressive aging of the population and the absence of curative treatments to date. In the United States, up to four million people suffer from Alzheimer's disease with no less than 100,000 deaths per year.

Although the mechanism and the origin of Alzheimer's disease are not yet fully elucidated, scientists were able to demonstrate two biological markers characteristic of it, which are amyloid plaques (or senile plaques) and neurofibrillar degeneration (or neurofibrillar tangles).

In detail, amyloid plaques consist of a peptide, called AP, which derives from a precursor protein called APP. As regards neurofibrillar degeneration, it consists of an accumulation, in the form of fibers and inside the neurons, of another protein, the Tau protein, which is then in abnormal form.

In connection with these markers, studies have shown that there is a correlation between the levels of soluble Aβ and the extent of synaptic loss and the severity of cognitive impairment in patients.

Therefore, any substance reducing Aβ neurotoxicity can be useful as a novel therapeutic agent for the treatment or prevention of Alzheimer's disease.

The only drugs existing today for treating Alzheimer's disease are of two types. Acetylcholinesterase inhibitors, such as donepezil, galanmine, and rivastigmine, and NMDA receptor antagonists such as memantine are the first type. However, these drugs only deal with the symptoms of Alzheimer's disease and only make it possible to moderately slow down the progression of the disease. In addition, taking these drugs is often accompanied by significant side effects such as nausea, diarrhea and hepatic disorders. There is therefore an urgent need to develop new alternatives.

The significant neuroprotective effect of AT000 extract in mice intoxicated with the Aβ 25-35 peptide was recently demonstrated. It has thus been demonstrated that the administration of this extract considerably reduces the harmful effects caused by this peptide such as learning deficit and oxidative stress (ISKANDAR et al., Chemistry of Advanced Materials, vol. 3(2); p:36-59, 2018).

This AT000 extract is derived from the extraction of an equal-mass mixture of plants consisting of Syzygium aromaticum, Santalum album, Aquilaria malaccensis, Boswellia carterii, Cyperus rotundus, Styrax benzoin, Liquidambar orientalis, Saussurea costus, and Dryobalanops aromatica.

Despite this fact, the efficacy of this extract against the deleterious effects induced by the Aβ 25-35 peptide, its use seems compromised since the plant Dryobalanops aromatica is not listed in the French pharmacopeia.

The inventors have surprisingly discovered that substituting the Dryobalanops aromatica plant with the plant Cinnamomum camphora made it possible to obtain an extract whose efficacy against the toxicity of the Aβ 25-35 peptide is similar to that of the AT000 extract while having a novel food-recognized composition.

### SUMMARY OF THE INVENTION

The inventors have identified three extracts that have a protective effect against the neurotoxicity of Aβ from a plan of formulation experiments based on the optimization of the antioxidant activity (DPPH test, activity of trapping the radicals of the extract) of different formulas based on 9 plants:
Extract 1a and 1b obtained from a mixture of different content levels of Syzygium aromaticum buds, of Santalum album heartwood, of Aquilaria malaccensis heartwood, of Boswellia carterii resin, of Cyperus rotundus buds, of Styrax benzoin resin, of Liquidambar orientalis resin, of Cinnamomum camphora leaves, and of Saussurea costus roots;
Extract 2 obtained from a mixture of different content levels of Syzygium aromaticum (clove) buds, of Santalum album heartwood, of Aquilaria malaccensis heartwood, of Boswellia carterii resin, of Cyperus rotundus buds, of Styrax benzoin resin, of Liquidambar orientalis resin, and Cinnamomum camphora leaves.

The present invention relates to a composition comprising extract 1a, 1b or 2.

Consequently, a first subject matter of the invention relates to a composition, which may be a pharmaceutical or nutraceutical composition, comprising an extract derived from a mixture of plants consisting of Syzygium aromaticum, Santalum album, Aquilaria malaccensis, Boswellia carterii, Cyperus rotundus, Styrax benzoin, Liquidambar orientalis, Cinnamrnomum camphora, and Saussurea costus.

The inventors have demonstrated that the last ingredient listed (Saussurea costus) made it possible to significantly reduce the effective dose of the extract as a whole.

Another object of the invention relates to a composition as defined above for use as a medication.

Another object relates to a composition as defined above for use in the treatment and/or prevention of a neurodegenerative disease, preferably Alzheimer's disease, in mammals, preferably in humans.

Another object of the invention relates to a food oil comprising at least 0.5% (by weight relative to the total weight of the oil) of a composition as mentioned above, preferably at least 1% or 2%.

A final object of the invention relates to a functional beverage produced by dilution of a glycerin macerate with water according to a ratio 1:10.

### BRIEF DESCRIPTION OF THE FIGURES

FIG. 1: Effect of different extracts and donepezil (DPZ) on a mouse model of the spatial working memory.

FIG. 2: Effect of different extracts and donepezil on a mouse model of the long-term contextual memory.

FIG. 3: Effect of different extracts and donepezil on lipid peroxidation.

FIG. 4: UHPLC-DAD analysis at 254 nm (a) of the extract 1b resulting from extraction by decoction of a mixture of plants in a hydroalcoholic mixture; (b) of the macerate 1b resulting from a maceration extraction of a mixture of plants in 100% glycerin; (c) of the macerate 1b resulting from a maceration extraction of a mixture of plants in a mixture of equal volumes of glycerin/ethanol.

FIG. 5: UHPLC-DAD analysis at 325 nm (a) of the extract 1b resulting from extraction by decoction of a mixture of plants in an hydroalcoholic mixture; (b) of the macerate 1b resulting from a maceration extraction of a mixture of plants in 100% glycerin; (c) of the macerate 1b resulting from a maceration extraction of a mixture of plants in a mixture of equal volumes of glycerin/ethanol.

FIG. 6: Lines of linearity of benzoic acid and of vanillin

FIG. 7: Study of the extraction kinetics: UHPLC-DAD analysis at 325 nm of the macerate 1b at different times T during the maceration in 100% glycerin.

FIG. 8: Neuronal life of a primary culture of cortical neurons treated with Aβ 1-42 followed by the composition according to the invention.

FIG. 9: Total network of neurites in a primary culture of cortical neurons treated with Aβ 1-42 followed by the composition according to the invention.

FIG. 10: Neuronal life of a primary culture of cortical neurons treated with Aβ 1-42 followed by DHA.

FIG. 11: Total network of neurites in a primary culture of cortical neurons treated with Aβ 1-42 followed by DHA.

### DETAILED DESCRIPTION OF THE INVENTION

The inventors have now developed a composition comprising an extract derived from a specific mixture of plants which makes it possible to protect against the neurotoxicity of Aβ.

The term “extract” refers to a substance extracted from a natural product, independently of its method of extraction or composition of the ingredients. For example, this includes those obtained by extracting soluble ingredients from a natural product using water, glycerin or an organic solvent, or those obtained by the extraction of specific ingredients, such as oil (e.g. olive oil). The extraction methods are well known to a person skilled in the art and consist in particular in a decoction, infusion or maceration of plant parts (buds, roots, leaves, etc.), ground or not, in an appropriate extraction solvent.

According to a preferred embodiment, the extract is obtained by decoction by means of a solvent selected from the group consisting of water, a linear or branched alcohol having 1 to 4 carbon atoms, ethyl acetate, dichloromethane, acetone, glycerin and mixtures thereof.

Advantageously, the extract is obtained by decoction by means of glycerin, a hydroalcoholic mixture or a glycerinated hydroalcoholic mixture, more preferably by means of a hydroalcoholic mixture.

According to another preferred embodiment, the extract is obtained by maceration of the mixture of plants at room temperature in 100% of glycerin or in an equal-volume mixture of glycerin and ethanol, preferably by maceration in 100% of glycerin. The extract thus obtained is also called macerate.

Preferably, the maceration of the mixture of plants in the glycerin is carried out between 30° C. and 50° C., preferentially at 40° C., for 30 minutes to 5 hours, preferentially between 1 and 4 hours, in particular 2 hours, preferentially in 100% of glycerin or in an equal-volume mixture of glycerin and ethanol, preferably by maceration in 100% of glycerin. The extract thus obtained is also called macerate.

Advantageously, the extract is not a dry extract, preferably the extract is obtained by maceration in glycerin, more preferably the extract is obtained by maceration in vegetable glycerin.

Dry extract is understood to mean a solid extract obtained after evaporation of the solvent that was used for its extraction.

Glycerin is understood to mean glycerin of vegetable origin or glycerin of animal origin, preferably glycerin of vegetable origin.

The term “hydroalcoholic mixture” is understood to mean a mixture of water and alcohol, preferably a mixture composed of 70% water and 30% alcohol.

The term “glycerinated hydroalcoholic mixture” is understood to mean a mixture of water, alcohol and glycerin, preferably a mixture composed of 35% water, 15% alcohol and 50% glycerin.

The term “decoction” is understood to mean an extraction method wherein the mixture of plants is placed in the presence of a solvent and then heated to the boiling point thereof for several hours before being filtered. The resulting filtrate constitutes the extract of the plant mixture. In the case of a mixture of solvent, the boiling point will be that of the azeotrope formed.

The term “maceration” is understood to mean an extraction method wherein the plant mixture is placed in the presence of a solvent at room temperature for several hours before being filtered. The resulting filtrate constitutes the extract of the plant mixture.

Clove (Syzygium aromaticum) is a species of plants of the Myraceae family and of the Syzygium genus. Clove trees originate from Indonesia, and their flowering buds form a spice called cloves. Preferably, the mixture of plants uses Syzygium aromaticum buds.

In the case where the extract does not comprise an equal-mass mixture of plants, the mixture has a content of between 25 and 55% (by weight relative to the total weight of the plant mixture) Syzygium aromaticum, preferably between 30 and 50%.

Indian sandalwood (Santalum album) is a tropical tree of the Santalaceae family and of the genus Santalum. It is the best-known source of sandalwood. This species mainly originates from Southern India, Sri Lanka, Australia, and the Malay Archipelago. Preferably, the mixture of plants uses Santalum album wood, and particularly preferably duramen (heartwood) of Santalum album.

In the case where the extract does not comprise an equal-mass mixture of plants, the mixture has a content of between 5 and 9% (by weight relative to the total weight of the plant mixture) Santalum album, preferably between 6 and 8%.

Aquilaria malaccensis, also known as aloe wood, eaglewood or agarwood, is a tree of the tropical forest belonging to the genus Aquilaria and is from the family of Thymelaeaceae. This species mainly originates from Bangladesh, Bhutan, India, Indonesia, Iran, Malaysia, Myanmar, the Philipppines, Singapore and Thailand. In the duramen, Aquilaria malaccensis produces a particular odorous resin in reaction to certain physical attacks (injuries, fire) or biological attacks (attacks of wood-eating insects, bacteria, and fungi). This resin is widely used in traditional medicine and in perfumery. Preferably, the mixture of plants uses Aquilaria malaccensis wood, and particularly preferably duramen (heartwood) of Aquilaria malaccensis.

In the case where the extract does not comprise an equal-mass mixture of plants, the mixture has a content of between 15 and 30% (by weight relative to the total weight of the plant mixture) Aquilaria malaccensis, preferably between 18 and 24%.

The frankincense tree, Boswellia carterii, belongs to the genus Boswellia and to the Burseraceae family. This tree mainly originates from Yemen and Somalia. The resin of this tree is one of the oldest known fragrant and medicinal resins worldwide. It has in particular been widely used in traditional Ayurvedic medicine. Preferably, the mixture of plants uses Boswellia carterii resin.

In the case where the extract does not comprise an equal-mass mixture of plants, the mixture has a content of between 5 and 9% (by weight relative to the total weight of the plant mixture) Boswellia carterii resin, preferably between 6 and 8%.

Nut grass (Cyperus rotundus) is a species of monocotyledonous plants of the Cyperaceae family. It is a herbaceous perennial plant with rhizomes and tubers. It is also known under the name of Java grass, nut grass, purple nut sedge, or red nut sedge. Originally from India, this species developed gradually from Africa to Southern Europe until it spread over a large part of the planet. The tubers are used for medicinal and food purposes. Preferably, the mixture of plants uses Cyperus rotundus buds.

In the case where the extract does not comprise an equal-mass mixture of plants, the mixture has a content of between 5 and 9% (by weight relative to the total weight of the plant mixture) Cyperus rotundus buds, preferably between 6 and 8%.

Styrax benzoin is a tree species originating from Sumatra, Indonesia, of the Styrax genus and belonging to the family of Styracaceae. This tree produces a balsamic resin (benzoin) whose odor is slightly vanilla-like. This resin is widely used in tropical Asia for its fragrance and its effect on wellness. Preferably, the mixture of plants uses Styrax benzoin resin.

In the case where the extract does not comprise an equal-mass mixture of plants, the mixture has a content of between 5 and 9% (by weight relative to the total weight of the plant mixture) Styrax benzoin resin, preferably between 6 and 8%.

Liquidambar orientalis, also known as oriental sweetgum, is a tree of the family Hamamelidaceae (or Altingiaceae depending on the phylogenetic classification). It is a wedge-leaf tree of the genus Liquidambar. In the vicinity of the region of the Eastern Mediterranean, this species occurs mainly in flood plains of southwestern Turkey and on the Greek island of Rhodes. Its resin is used in both incense and perfume. Preferably, the mixture of plants uses Liquidambar orientalis resin.

In the case where the extract does not comprise an equal-mass mixture of plants, the mixture has a content of between 5 and 9% (by weight relative to the total weight of the plant mixture) Liquidambar orientalis resin, preferably between 6 and 8%.

Camphorwood, camphor tree, Shiu wood, or camphor laurel are the various names of Cinnamomum camphora, a species of trees of the family of Lauraceae originating from China, Taiwan, and Japan, which has become rather common across the other continents. The leaves and shoots are distilled to obtain essential oils containing camphor. Preferably, the mixture of plants uses Cinnamomum camphora leaves.

In the case where the extract does not comprise an equal-mass mixture of plants, the mixture has a content of between 5 and 9% (by weight relative to the total weight of the plant mixture) Cinnamomum camphora leaves, preferably between 6 and 8%.

Saussurea costus is a large robust herbaceous plant of the Asteraceae family. Originally from Asia (Himalayas, Kashmir, India, Pakistan), it is often grown for its medicinal properties. Preferably, the mixture of plants uses roots of Saussurea costus.

In the case where the extract does not comprise an equal-mass mixture of plants, the mixture has a content of between 5 and 9% (by weight relative to the total weight of the plant mixture) Saussurea costus roots, preferably between 6 and 8%.

Typically, the content of extract in the composition according to the invention is between 0.1% and 90% by weight relative to the total weight of the composition.

This range is justified by the forms that the composition can take according to the invention.

Thus, in the context of a syrup (concentrate) or a gelcap, the content of extract in the composition will be between 20% and 50% by weight relative to the total weight of the composition.

Now, in the context of a food oil or a functional beverage, the content of extract in the composition will instead be between 0.1% and 10% by weight relative to the total weight of the composition, more preferably between 0.5% and 5% by weight relative to the total weight of the composition.

In any case, the composition can allow daily administration of an extract of the mixture of plants from 62.5 mg in mice and from 625 mg in humans.

Preferably, the composition according to the invention is characterized in that the extract is obtained from a mixture of plants which has:
a content of between 25 and 55% of Syzygium aromaticum,
a content of between 5 and 9% of Santalum album,
a content of between 15 and 30% of Aquilaria malaccensis,
a content of between 5 and 9% of Boswellia carterii,
a content of between 5 and 9% of Cyperus rotundus,
a content of between 5 and 9% of Styrax benzoin,
a content of between 5 and 9% of Liquidambar orientalis,
a content of between 5 and 9% of Cinnamomum camphora; and
a content of between 5 and 9% of Saussurea costus.

The percentage of each plant used in the extract corresponds to the percentage by weight relative to the total weight of the mixture of plants.

The composition according to the invention may be in the form of dry solids, resin, emulsion or liquid form.

More specifically, the composition according to the invention may be in the form of flavored oil, syrup, tablets, capsules, powder, gelcaps, sticks, pouches, bulbs, droppers or in injectable form.

According to a particular embodiment, the composition according to the invention is in the form of a gelcap, an oil or a functional beverage

Particularly preferably, the gelcap is a “plant-based” gelcap.

Such a gelcap, in particular a plant-based gelcap, can be carried out simply, in particular with a cellulose-based coating Hydroxypropylmethylcellulose, or HPMC, also known as “Hypromellose”); to the cellulose, natural dyes may be added, so as to obtain the coating having the desired properties.

Typically, the composition according to the invention has a proportion of Hydroxypropylmethylcellulose comprised between 50 and 150 mg, for example between 75 and 125 mg.

For the gelcap as such, its weight is ideally between 0.25 and 0.75 gram/gelcap to benefit from an easily ingestible gelcap and, consequently, consistent usage by the subject.

Said coating may also comprise opacifying agents.

Other pharmaceutically and/or acceptable agents may be added, such as antioxidants, bulking agents, fluidizers, natural extracts, minerals, trace elements, amino acids, fatty acids, anti-caking agents, natural oils, flavors, dyes, acidifiers, thickeners, preservatives and sweeteners.

As examples of antioxidants, mention may be made of polyphenols, in particular in the form of plant extracts (green tea, grape tea, ginseng tea extracts), vitamin C, in particular in the form of plant extracts (extract of acerola, pomegranate, citrus fruit), or vitamin E, in particular in the form of plant extracts; or derivatives thereof.

As examples of filler agents, mention may be made of microcrystalline cellulose, potato maltodextrin, or magnesium lactate, preferably microcrystalline cellulose.

As examples of fluidizers, mention may be made of magnesium silicate, magnesium stearate, or colloidal silica.

As examples of minerals or trace elements, mention may be made of magnesium, iodine, iron, copper, zinc, selenium, chromium, molybdenum, manganese, silicon, vanadium, nickel or tin.

As examples of amino acids, mention may be made of alanine, cysteine, aspartic acid, glutamic acid, phenylalanine, glycine, histidine, isoleucine, lysine, leucine, methionine, asparagine, proline, glutamine, arginine, serine, threonine, valine, tryptophan or tyrosine.

As examples of fatty acids, mention may be made of unsaturated fatty acids such as omega-3 or omega-6 acids.

Advantageously, the composition according to the invention comprises at least one fatty acid belonging to the omega-3 family.

Preferably, the composition according to the invention comprises docosahexaenoic acid (DHA) which is an unsaturated fatty acid belonging to the omega-3 family

Preferably, the DHA content in the composition is adjusted so as to allow daily administration of 250 mg, which constitutes the recommended dose.

As examples of anti-agglomerating agents commonly used in the food industry, mention may be made of magnesium stearate (E470b), silicon dioxide (E551) and colloidal silica.

As examples of thickening agent, mention may be made of potato starch, hydroxypropylmethylcellulose, citrus pectin, guar gum, carob, agar-agar, konjac, hydrogenated oils or beeswax.

As examples of acidifiers, mention may be made of citric acid.

As examples of sweeteners, mention may be made, among other things, of xylitol, aspartame, glucose syrup, fructooligosaccharide syrup, maltitol in powder or syrup form, acesulfame potassium, fructooligosaccharide and sodium cyclamate.

As dyes, mention may be made of curcumins (E100), carminic acid (E120), erythrosine (E127), chlorophyll and chlorophyllins (E140), copper-chlorophyll and chlorophyllins complexes (E141), caramel (E150), carotenoids (E160), anthocyanins (E163), calcium carbonate (E170), iron oxide and hydroxide (E172), or orcein (E182).

As examples of preservatives, mention may be made of potassium sorbate, sodium benzoate or ascorbyl palmitate (antioxidant).

Now, all these compounds are in no way limiting with respect to pharmaceutically acceptable agents that can be added to the composition according to the invention, and other agents can be envisaged.

In the context of a nutraceutical application, the composition as defined above can be used in the manufacture of a food oil and/or a functional beverage.

Also, according to a particular embodiment, the composition according to the invention is a food oil (for example olive oil) or a functional beverage.

In this context, the composition according to the invention has a content of at least 0.5% (by weight relative to the total weight) of a composition as defined above, preferably at least 1% or 2%.

Food oil is understood to mean any oil capable of being used in food, namely olive oil, sesame, nut, rapeseed, seed of reasons, sunflower or even mixtures thereof.

The term “functional beverage” is understood to mean any non-alcoholic beverages that allow the consumer to be hydrated but also to improve their well-being and health, or can even reduce the risk of diseases. These are, in particular, sports drinks, energy drinks, smart drinks, ready-to-drink (RTD) coffees and teas, improved water.

In the context of the use of a composition as defined as a medication, it is administered in a pharmaceutically acceptable vehicle.

The term “pharmaceutically acceptable vehicle” is understood to mean any vehicle which does not interfere with the efficacy of the biological activity of the composition or extract according to the invention and which is not toxic to the host to whom the composition or extract according to the invention is administered.

In connection this time with such a composition for preventing and/or treating a neurodegenerative disease in a subject, it is preferably a mammal and, particularly preferably, a human.

Regarding neurodegenerative diseases, it is advantageously Alzheimer's disease, Parkinson's disease, Huntington's disease and amyotrophic lateral sclerosis.

The examples below are provided to illustrate the embodiments of the invention and should not be construed to limit the scope of the invention.

### EXAMPLE

The invention is illustrated using examples.

### Example 1

An in vivo study was carried out in a mouse model of Alzheimer's disease, to evaluate the neuroprotective effect of plant extracts AT000, 1a, 1 b, and 2, obtained by decoction in a hydroalcoholic mixture, against the toxicity induced by the Aβ 25-35 peptide.

### Materials

### Plant Extracts

### A—Extraction Method

### (a) Extract AT000 Obtained by Decoction of a Mixture of Plants in a Hydroalcoholic Medium

A total of 175 g of a pre-ground equi-mass mixture of Syzygium aromaticum buds, duramen (heartwood) of Santalum album, duramen (heartwood) of Aquilaria malaccensis, Boswellia carterii resin, Cyperus rotundus buds, Styrax benzoin resin, Liquidambar orientalis resin, Saussurea costus root and Dryobalanops aromatica resin is extracted with the equivalent of 10 volumes of a water/ethanol mixture (70:30, v/v) at 80-85° C. using a condenser at reflux for 3 h. The extract is then filtered under vacuum using a Buchner flask at 60° C. A resin is thus obtained.

### (b) Extract 1a Obtained by Decoction of a Mixture of Plants in a Hydroalcoholic Medium

A total of 175 g of a pre-ground equi-mass mixture of Syzygium aromaticum buds, duramen (heartwood) of Santalum album, duramen (heartwood) of Aquilaria malaccensis, Boswellia carterii resin, Cyperus rotundus buds, Styrax benzoin resin, Liquidambar orientalis resin, Saussurea costus root and Cinnamomum camphora leaves is extracted with the equivalent of 10 volumes of a water/ethanol mixture (70:30, v/v) at 80-85° C. using a condenser at reflux for 3 h. The extract is then filtered under vacuum using a Büchner flask for 3 h. A resin is thus obtained.

### (c) Extract 1 b Obtained by Decoction of a Mixture of Plants in a Hydroalcoholic Medium

Extract 1 b is obtained according to a protocol identical to that making it possible to obtain extract 1a, with the difference that 5 equivalents of buds of the Syzygium aromaticum plant and 3 equivalents of duramen (heartwood) of the Aquilaria malaccensis plant are used instead of an equivalent. The other plants are used as above at an equivalent level. A resin is thus obtained.

### (d) Extract 2 Obtained by Decoction of a Mixture of Plants in a Hydroalcoholic Medium

Extract 2 is obtained according to a protocol identical to that making it possible to obtain extract 1b (A-c), except that the plant mixture making it possible to obtain extract 2 does not contain Saussurea costus. A resin is thus obtained.

### (e) Extract 1 b Obtained by Maceration of a Mixture of Plants in 100% Vegetable Glycerin

The macerate 1 b is obtained by maceration from Syzygium aromaticum buds (5 equiv.), duramen (heartwood) of Santalum album (1 equiv.), duramen (heartwood) of Aquilaria malaccensis (3 equiv.), Boswellia carterii resin (1 equiv.), Cyperus rotundus buds (1 equiv.), Styrax benzoin resin (1 equiv.), Liquidambar orientalis resin (1 equiv.), Saussurea costus roots (1 equiv.), and Cinnamomum camphora leaves (1 equiv.) in 100% vegetable glycerin at room temperature for 3 h away from light. The mixture is extracted with the equivalent by weight of 1 mass of dried and ground plants per 4 volumes of vegetable glycerin (ratio 1:4). The macerate is regularly mixed. The extract is then filtered under vacuum using a Büchner flask.

### (f) Extract 1b Obtained by Maceration of a Mixture of Plants in an Ethanol/Glycerin Mixture

The protocol is identical to point (A-e) except for the maceration solvent which is composed of an ethanol/vegetable glycerin mixture with a volume ratio of 50/50 and of the same plant-solvent mass ratio of 1:4.

### B—Determination of the Chemical Profile of the Different Extracts 1b

### 1—Preparation of Control Solutions

A stock solution was prepared: 10.0 mg of control were placed in a 10 mL volumetric flask. After solubilization in approximately 3 mL of methanol using ultrasound, the solution was adjusted to the line of the gauge with the same solvent. The dilutions were prepared at 100, 20, 10, 5 and 1 μg/mL. Each dilution was filtered in a 0.22 μm filter before being injected.

### 2—Preparation of the Samples to be Analyzed

### (a) Dry Extract 1 b Obtained by Decoction in a Hydroalcoholic Medium According to Protocol A-(c)

5.1 mg of extract were solubilized in 2.55 mL of 30% EtOH (v/v) using ultrasound to obtain a solution at a final concentration of 2 mg/mL. 2 mL of this solution were filtered on a 0.22 μm filter before being injected.

### (b) Extract (or Macerate) 1b Obtained by Maceration in 100% Glycerin According to Protocol A-(e)

The glycerin extract was diluted to one-tenth with methanol. 100 μL of the extract were mixed with 900 μL of methanol using a vortex. 2 mL of this solution were filtered on a 0.22 μm filter before being injected.

### (c) Extract (or Macerate) 1b Obtained by Maceration in an Ethanol/Glycerin Mixture According to Protocol A-(f)

The glycerin extract was diluted to one-tenth with ethanol. 100 μL of the extract were mixed with 900 μL of ethanol using a vortex. 2 mL of this solution were filtered on a 0.22 μm filter before being injected.

### 3—Analysis Method

The chemical profile of the various extracts 1b was determined by means of an analysis by ultra-high performance liquid chromatography equipped with a diode array UV detector and coupled to a mass spectrometer. A Kinetex Polar C18 column (150×4.6 mm; 2.6 μm) is used for the separation of compounds. The mobile phase consists of water acidified to 0.1% (volume/volume) by formic acid as solvent A and acetonitrile acidified to 0.1% (volume/volume) by formic acid as solvent B. The elution gradient is following: 0-2 min 15% B, 2-14 min 15-100% B, 14-17 min 100% B, 17-17.01 min 100-15% B. 17.01-20 min 15% B. The flow rate and the column oven are set at 0.5 mL/min and 40° C., respectively.

The analysis is carried out from 2 μL of the samples to be analyzed.

### 4—Results and Discussion

### (a) Qualitative Analysis

The chromatograms obtained us information on the diversity of the molecules present in the extract 1 b from the different types of extraction A-(c), A-(e), A-(f).

Qualitatively, the three extracts 1b have a similar chromatographic profile. At 254 nm, an additional peak corresponding to methyl cinnamate is nevertheless observed only on the chromatogram of the extract 1b resulting from a maceration in 100% glycerin (A-(e)) (FIG. 4 (b)).

The inventors were also able to identify 5 other compounds common to these three extracts 1b: isobiflorin, biflorin, vanillin, benzoic acid and eugenol (FIG. 4 (b)).

(b) Quantitative Analysis of Vanillin and Benzoic Acid in the Extract 1b from the Different Extraction Types A-(c), A-(e), A-(f.

The detection of benzoic acid and vanillin was carried out at their maximum absorption wavelength, which is respectively 270 nm and 325 nm.

In order to quantify the content of each compound in the dry extract and the concentration in the liquid extracts, a calibration curve according to the model log 10(Y)=a×log 10(X)+b was carried out.

The calibration curve of the two standards is linear (FIG. 6) with correlation coefficients (R2) greater than 0.999. The back-calculated concentration deviations are less than 5%.

Using the equation of the calibration straight line of each standard, the concentration and content of benzoic acid and of vanillin present in the extract was calculated.

The results are shown in Table 1 below.

TABLE 1
Quantitative estimate of the benzoic acid and vanillin in the various
extracts 1b obtained according to protocols A-(c), A-(e) and A-(f)

| | Extract 1b | | |
|---|---|---|---|
| | A-(c) | A-(e) | A-(f) |
| Vanillin | 0.8%^{a} | 1.5 mg/mL^{b} | 1.2 mg/mL^{b} |
| Benzoic acid | 1.7%^{a} | 2.7 mg/mL^{b} | 3.0 mg/mL^{b} |

^{a}results expressed as % mass/mass;
^{b}results expressed in concentration of the compound in 1 mL of the liquid extract.
(c) Study of the extraction kinetics in 100% glycerin

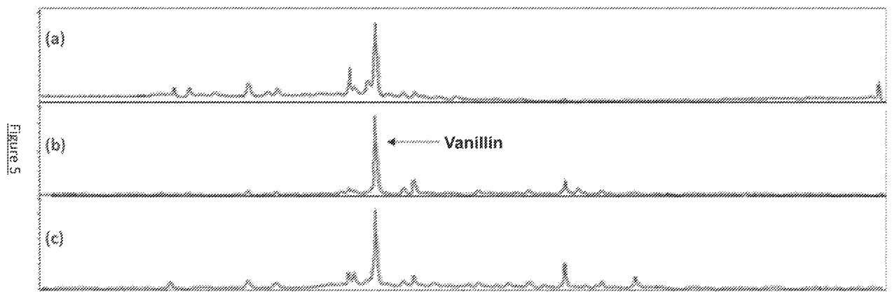

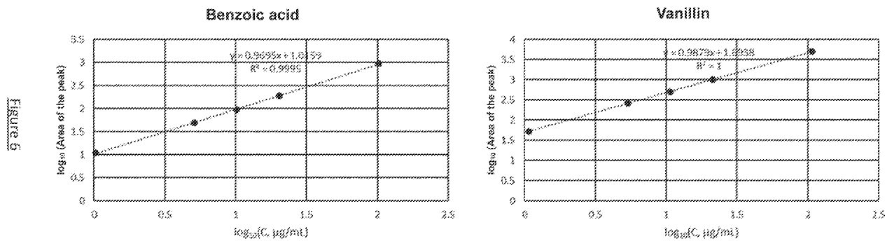

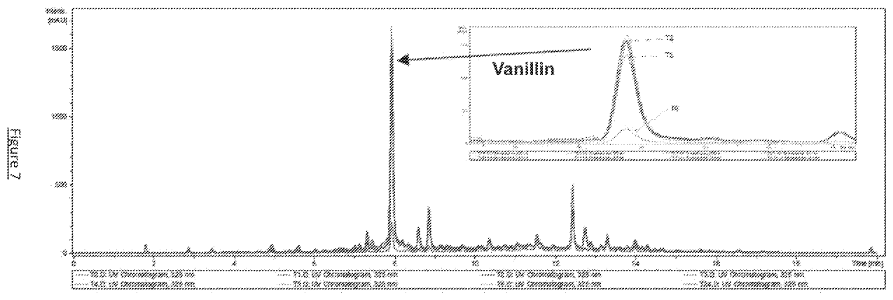

The glycerin macerates sampled at T0, T1h, T2h, T3h, T4h, T5h, T6h and T24h were prepared according to the preparation method described in part B.2(b) and are analyzed according to the analysis method described in part B3-. The results of the chromatographic profiles at 325 nm of the various samples are presented in FIG. 7.

These results indicate that the maximum concentration of vanillin is reached after 2 h of maceration.

### Animals

Male Swiss mice, 6 weeks old and weighing 30-35 g, originating from JANVIER LABS are used throughout the study. The mice are housed in a group with free access to food and water except during behavioral experiments. The mice are held in an animal house with controlled temperature and humidity, under a light/dark cycle of 12 h (light turned off at 19:00). The mice are numbered by marking their tail with a permanent marker. All animal procedures are carried out in strict compliance with the European Union Guidelines of 22 Sep. 2010 (2010/63/EU).

### 2. Treatment Method

### a) Procedure for Administration of the Aβ 25-35 Peptide and the Mutated Aβ 25-35 Peptide

A homogeneous oligomeric preparation of the Aβ 25-35 peptide and the mutated Aβ 25-35 peptide (negative control) was carried out according to a procedure belonging to AMYLGEN. The preparations were dissolved in sterile distilled water at a concentration of 3 mg/mL, then stored at −20° C. until use. Before the injection, the peptides were aggregated by incubation at 37° C. for 4 days. Each mouse is anesthetized 5 min with 2.5% isoflurane before receiving, by means of a 26-gauge stainless steel syringe, gradual injection, for a duration of 30 s, 3 μL of the Aβ 25-35 peptide (9 mmol/mouse) or the mutated and inactive Aβ 25-35 peptide (9 mmol/mouse) in the right lateral ventricle of the brain according to a method already described. The syringe needle is held at the injection site for an additional period of 30 s before being removed.

At D1, the mice received, at 10:00, a single intracerebroventricular injection of 3 μL (3 mg/mL) of the oligomeric peptide Aβ 25-35 or the mutated Aβ 25-35 peptide (negative control)

### b) Procedure for Administering Donepezil (Reference) and Plant Extracts AT000, 1a, 1b and 2 Obtained by Decoction in a Hydroalcoholic Mixture.

On Day −14 (D−14) on Day +10 (D10), the vehicle (5% solution of DMSO in water), the extracts AT000, 1a, 1b, or 2 are administered to mice twice a day by feeding (per os), once at 9:00 and once at 17:00. Each extract is solubilized in an aqueous 5% DMSO solution and is freshly prepared just before each oral administration.

Donepezil (reference) is also administered orally (1 mg/kg) but only once per day (9:00), from D−14 to D10, after having been dissolved beforehand in water.

The volume of solution administered per mouse is calculated as a function of the individual weight of each mouse (5 mL/kg).

At D8, all the animals are tested for their spontaneous alternation behavior in the Y-maze, an indicator of spatial working memory

The long-term non-spatial memory of the mice is evaluated by means of a passive avoidance procedure that takes place in two stages. The 1st part of the test takes place on D9 and is a so-called learning phase. The 2nd part takes place on D10 and is a so-called retention phase.

### c) Euthanasia of the Mice

At D10, immediately after the passive avoidance retention session, the mice are decapitated. The hippocampus and the cortex of each group of mice are quickly taken, weighed and stored in liquid nitrogen until analysis.

### Animals and Treatment Groups

Seventy-eight male Swiss mice of 30 to 35 g are used for this study and were randomly distributed in the groups

Eight groups of mice were created for this study (Table 2):

TABLE 2

| Animal | | Number |
|---|---|---|
| group | Treatment administered | of mice |
| 1 | Mutated Aβ₂₅₋₃₅ (3 μL, 3 mg/mL) + vehicle | 10 |
| (negative | (double-distilled sterile water) | |
| control) | | |
| 2 | Aβ₂₅₋₃₅ (3 μL, 3 mg/mL) + vehicle (double- | 10 |
| | distilled sterile water) | |
| 3 | Aβ₂₅₋₃₅ (3 μL, 3 mg/mL) + vehicle (double- | 10 |
| | distilled sterile water) Extract AT000 (125 mg/kg | |
| | b.i.d.) + vehicle (5% DMSO) | |
| 4 | Aβ₂₅₋₃₅ (3 μL, 3 mg/mL) + vehicle (double- | 10 |
| | distilled sterile water) Extract 1a (125 mg/kg | |
| | b.i.d.) + vehicle (5% DMSO) | |
| 5 | Aβ₂₅₋₃₅ (3 μL, 3 mg/mL) + vehicle (double- | 10 |
| | distilled sterile water) Extract 1b (125 mg/kg | |
| | b.i.d.) + vehicle (5% DMSO) | |
| 6 | Aβ₂₅₋₃₅ (3 μL, 3 mg/mL) + vehicle (double- | 10 |
| | distilled sterile water) Extract 2 (125 mg/kg | |
| | b.i.d.) + vehicle (5% DMSO) | |
| 7 | Aβ₂₅₋₃₅ (3 μL, 3 mg/mL) + vehicle (double- | 10 |
| | distilled sterile water) Extract 1b (31.25 mg/kg | |
| | b.i.d.) + vehicle (5% DMSO) | |
| 8 | Aβ₂₅₋₃₅ (3 μL, 3 mg/mL) + vehicle (double- | 8 |
| (reference) | distilled sterile water) Donepezil (1 mg/kg) + | |
| | vehicle (water) | |

* b.i.d. (bis in die): administration twice daily

### 3. Test Procedure—Tested Parameters

### Spontaneous Alternation Behavior Test in a Y-Maze

At D8, all the mice are tested for their spontaneous alternation behavior in the Y-maze, an indicator of spatial working memory. The Y-maze is made of gray polyvinyl chloride.

Each arm measures 40 cm long, 13 cm high, 3 cm wide at bottom, 10 cm wide at top and converges at an equal angle.

Each mouse is placed at the end of one arm and is allowed to move freely in the maze during a session of 8 minutes.

The series of arm entries, including possible returns to the same arm, are checked visually.

An alternation is defined as entries into the three arms on consecutive occasions. The maximum number of alternations is therefore the total number of arm entries minus two and the alternation percentage is calculated as follows:

[(actual alternations)/(maximum alternations)]×100.

The parameters include the alternation percentage (memory index) and the total number of arm entries (exploration indexes).

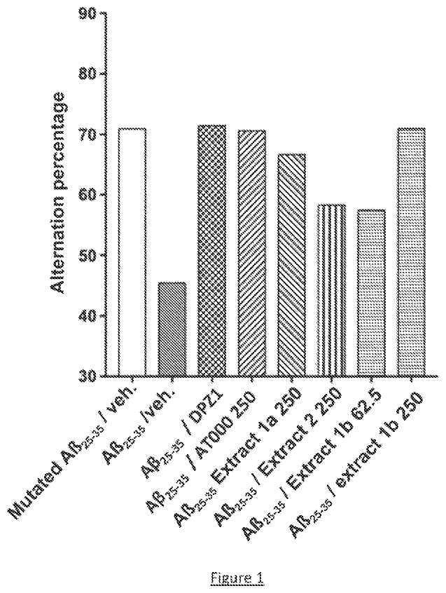

Mice that have an extreme behavior (alternation percentage <20% or >90% or a number of arm entrances <10) are excluded from the calculations. In this case, no animal was excluded. The results are shown in FIG. 1.

### Passive Avoidance Test

On D9 and D10, all the mice were tested in the progressive passive avoidance task. This test makes it possible to evaluate the long-term non-spatial memory.

The apparatus of the test consists of a box with two compartments (15×20×15 cm in height), one of which is illuminated by white walls of polyvinyl chloride and the other obscured by black walls made of polyvinyl chloride and having a grid on the ground. A guillotine door separates the two compartments. A 60 W lamp positioned at 40 cm above the appliance illuminates the white compartment during the experiment. At the gate, random electrical shocks of 0.3 mA are delivered to the feet for 3 seconds by virtue of a random electrical generator (Lafayette Instruments, Lafayette, USA).

The 1st phase of the experiment, called learning or training, takes place on D10. The guillotine door is initially closed during the training session. Each mouse is placed in the white compartment. After 5 seconds, the door rises. When the mouse enters the dark compartment and places all of its feet on the grid, the door closes and the random electrical shock is delivered to the feet for 3 seconds. The latency time before entering the dark compartment and the number of vocalizations are recorded. The number of vocalizations does not differ between the groups indicating that all the animals also received the electric shock

The animals for which the latency time during the training session is less than 10 seconds are discarded from the experiment.

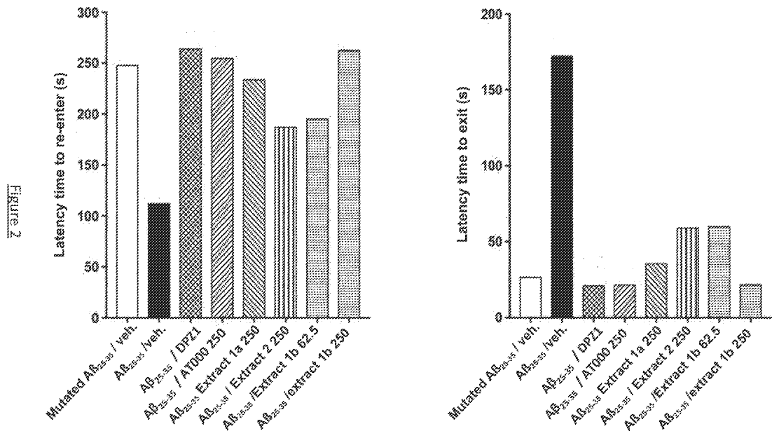

The 2nd phase of the experiment, called retention, is carried out 24 hours after the 1st phase. Each mouse is put back in the white compartment. The door separating the two compartments is raised after 5 seconds. The latency time for entering the dark compartment is recorded for a duration of 300 seconds. The number of entries and the escape time, that is, the time taken to return to the white compartment, are measured for 300 seconds. Animals that exhibit latencies during the retention session of less than 10 seconds are taken out of the experiment. The results are shown in FIG. 2.

### Oxidative Stress Test—Measurement of Lipid Peroxidation

Six hemi-hippocampus of each group of mice are thawed for analysis. The hippocampus are then homogenized in cold methanol ( 1/10 w/v), centrifuged at 1000 g for 5 min and the supernatant is placed in EPPENDORF tubes. The reaction volume of each homogenate is added to a solution comprising 1 mM FeSO4, 0.25 M H2SO4, xylenol orange 1 mM and incubated for 30 min at room temperature. After reading the absorbance at 580 nm (A580 1), 10 μL of cumene hydroperoxide at 1 mM (CHP) are added to the sample before being incubated for 30 min at room temperature, to determine the maximum oxidation level. The absorbance is measured at 580 nm (A580 2).

The level of lipid peroxidation is determined in CHP equivalents according to the following equation:

$$CHPE = A 5801 / A 5802 \times [ CHP ( n mol ) ]$$

And is expressed in CHP equivalents per fabric weight and as a percentage of the control group data.

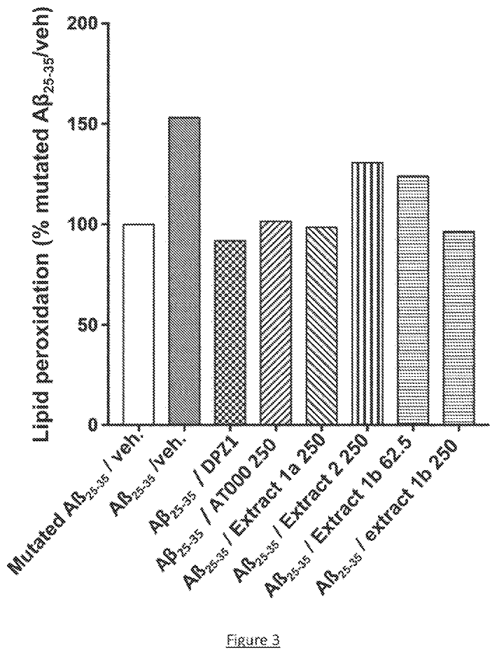

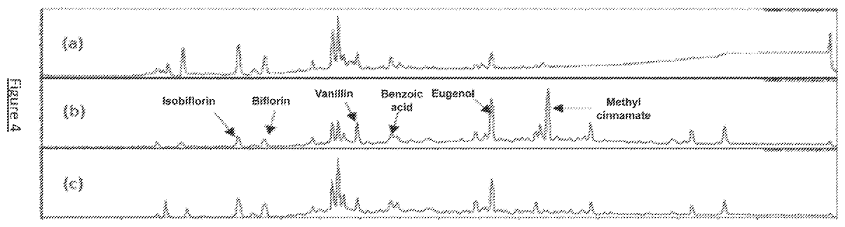

The results are shown in FIG. 3.

### 4. Results and Discussion

### 2.1 Spontaneous Alternation Behavior

The spatial working memory is evaluated by means of a spontaneous alternation test in the Y-maze.

As shown in FIG. 1, the injection of the Aβ 25-35 peptide greatly alters the spatial working memory of the mice compared to that of the mice which received only the injection of the mutated Aβ 25-35 peptide (negative control)

This deleterious effect is significantly avoided by daily administration of 1 mg/kg of Donepezil (DPZ) according to the method described above

The results also show that plant extracts AT000 and 1b induce a neuroprotective effect comparable to that of Donepezil when a daily dose of 250 mg/kg of one of these extracts is administered to mice intoxicated as described in the treatment method defined above. Administration in mice intoxicated with a lower dose of extract 1b (62.5 mg/kg versus 250 mg/kg) induces a significant reduction, but less so (22%) for spontaneous alternation behavior.

The results also show that extract 1a almost counteracts the deficit of spatial working memory induced by the injection of the Aβ 25-35 peptide. Indeed, this extract allows virtually complete protection (93%) of the cognitive performance of the intoxicated mice.

Finally, only a slight improvement in performance is observed with extract 2 (extract 2: 78% vs Aβ 25-35: 64%). However, it would seem that the plant Saussurea costus is essential in the composition of the extract to obtain optimal protection of cognitive performance of the intoxicated mice (to 250 mg/kg, extract 2: 78% vs extract 1b 100%).

### 2.2 Progressive Passive Avoidance Test

Long-term contextual memory was evaluated by means of a passive avoidance test.

The results show that the Aβ 25-35 peptide produces a significant decrease in the performance of memory in mice intoxicated as shown in FIGS. 2a and 2b compared to the negative control.

This deleterious effect is significantly avoided by daily administration of 1 mg/kg of Donepezil (DPZ) according to the method described above.

It is observed that plant extract AT000 and plant extract 1b induce a neuroprotective effect comparable to that of Donepzil when a daily dose of 250 mg/kg of one of these extracts is administered to mice intoxicated according to the treatment method defined above. Now, administration in mice intoxicated from a lower dose of the extract 1b (62.5 mg/kg versus 250 mg/kg) slightly reduces the efficacy of this extract on the deleterious effects of the Aβ 25-35 peptide, nevertheless this efficacy is of the same order of magnitude as that of extract 2 when the extract is administered at 250 mg.

It is also observed that the extract 1a strongly mitigates the long-term contextual memory deficits induced by the Aβ 25-35 peptide.

Finally, the administration of extract 2 allows a slight improvement in the behavior of mice intoxicated with the Aβ 25-35 peptide. It should be noted that the differences in the entry and exit latency times observed between the extracts 1b and 2 underline the importance of the plant Saussurea costus in the plant mixture making it possible to obtain the extract with the best performance.

### 2.3 Measurement of Lipid Peroxidation

The measurement of the lipid peroxidation of the hippocampus of the intoxicated mice constitutes an indicator of the level of oxidative stress in this organ.

The results show that the mice intoxicated with Aβ 25-35 peptide have a significant increase in lipid peroxidation in the hippocampus (+50% compared with the negative control, FIG. 3).

Now, it is observed that this oxidative stress is completely avoided by the AT000, 1a or 1 b extracts when they are administered at a daily dose of 250 mg/kg as defined above in the treatment method. These extracts have a neuroprotective effect against the toxicity of Aβ 25-35 peptide comparable to that of Donepezil (positive control), when the latter is administered at a daily dose of 1 mg/kg as described in the treatment method defined above.

It should be noted that the treatment of mice intoxicated with a lower daily dose of the extract 1b (62.5 mg/kg vs 250 mg/kg) shows an increase of 20% of the lipid peroxidation relative to the negative control.

Regarding extract 2, the results show that it would slightly wait the oxidative stress: an increase of 30% of the lipid peroxidation is observed against 50% in mice intoxicated with the Aβ 25-35 peptide and not treated. This result again emphasizes the importance of the plant Saussurea costus in the composition of the extract to obtain optimal protection of cognitive performance of mice intoxicated with the Aβ 25-35 peptide.

### CONCLUSION

The alteration of the memory is the early characteristic of Alzheimer's disease and these results clearly show that the toxic effect of the amyloid peptide Aβ 25-35 on behavioral and cognitive performance (including the memory) is avoided or mitigated by extracts 1a, 1b and 2 of the invention obtained by decoction in a hydroalcoholic mixture.

### Example 2

### Experimental Methods and Design

All experiments will be carried out in accordance with the Guide for the Care and Use of Laboratory Animals of National Research Council (US) and will follow current European Union regulations (directive 2010/63/EU). Approval number: B1301310.

### Primary Culture of Cortical Neurons

The rat cortical neurons will be cultured as described by Callizot et al., 2013; 2020. The 15-day gestation female (Wistar) rats will be killed using deep anesthesia with a C02 chamber and cervical dislocation. Briefly, the fetuses will be collected and immediately placed in the chilled Leibovitz's L15 medium with a solution of penicillin (10,000 U/mL) and of streptomycin (10 mg/mL) at 2% (PS) and 1% of bovine serum albumin (BSA). The cortex will be treated for 20 min at 37° C. with a trypsin-EDTA solution at a final concentration of 0.05% trypsin and 0.02% EDTA. Dissociation will be stopped by adding Dulbecco's modified Eagle medium (DMEM) with 4.5 g/L of glucose, containing DNAse I grade II (final concentration 0.5 mg/mL) and 10% fetal calf serum (FCS). The cells will be mechanically dissociated by three forced passages through the end of a 10 mL pipette. The cells will then be centrifuged at 515×g for 10 minutes at 4° C. The supernatant is removed, and the pellet is resuspended in a culture medium composed of Neurobasal medium with a solution of 2% of supplement B27, 2 mmol/liter of L-glutamine, 2% PS solution and 10 ng/mL of brain-derived neurotrophic factor (BDNF). The viable cells will be counted in a Neubauer cytometer, using the trypan blue exclusion test. The cells will be seeded at a density of 25,000 per well in 96-well plates pre-coated with poly-L-lysine and will be cultivated at 37° C. in an air incubator (95%)—C02 (5%). The medium will be changed every 2 days.

To avoid the edge effect, the first and last columns as well as the first and last lines of the plate will not be used in the study. The empty wells will be filled with water.

Applying the Tested Compounds and Chronic Injury with Aβ 1-42

Vehicle: Culture medium (up to 0.1% glycerol).

Pre-treatment: On day 11 of the culture, the tested compounds (Composition according to the invention, DHA, BDNF) will be dissolved in the culture medium (0.1% maximum glycerol) and incubated for 1 hour before the injury.

Injury: The preparation of Aβ 1-42 will be carried out according to the procedure described by Callizot et al., 2020. Briefly, the peptide Aβ 1-42 (Bachem, 1071428) will be dissolved in the defined culture medium mentioned above, at an initial concentration of 20 μM. This solution will be gently stirred for 3 days at 37° C. in the dark and immediately used after having been correctly diluted in the culture medium at the concentrations used (15 μM, 2 μM of oligomers).

At day 11 of the culture, the cortical neurons will be injured with a solution of Aβ 1-42. The preparation of Aβ 1-42 will be added to a final concentration of 15 μM (2 μM of oligomer, APO) diluted in the control medium in the presence of the compounds, for 24 hours.

### Organization of Culture Plates

The compounds tested will be tested on two cultures in 96-well plates (n=6 culture wells per condition).

### Efficacy of the Composition According to the Invention and DHA

TABLE 3

| Plate 1 -pre-incubation 1 h | Plate 2 -pre-incubation 1 h |
|---|---|
| Control | Control |
| Aβ 1-42 (15 μM)/Control | Aβ 1-42 (15 μM)/Control |
| +Composition according to the invention | +DHA (10 nM) |
| (0.01 μg/ml) | |
| +Composition according to the invention | +DHA (30 nM) |
| (0.03 μg/ml) | |
| +Composition according to the invention | +DHA (0.1 μM) |
| (0.1 μg/ml) | |
| +Composition according to the invention | +DHA (0.3 μM) |
| (0.3 μg/ml) | |
| +Composition according to the invention | +DHA (1 μM) |
| (1 μg/ml) | |
| +Composition according to the invention | +DHA (3 μM) |
| (3 μg/ml) | |
| +Composition according to the invention | +DHA (10 μM) |
| (7.5 μg/ml) | |
| +BDNF (50 ng/mL) | +BDNF (50 ng/mL) |

### Assessment of End Point

### Immunolabelin MAP2 and AT100

24 hours after the injury, the cortical neurons will be fixed by a cold solution of ethanol (95%) and acetic acid (5%) for 5 min at −20° C. The cells will be washed twice in PBS, then permeabilized. The nonspecific sites will be blocked with a solution of PBS containing 0.1% saponin and 1% FCS for 15 min at room temperature. The cultures will be incubated with a polyclonal chicken anti-microtubule-associated protein 2 (MAP-2) antibody at a dilution of 1/1000 in PBS containing 1% fetal calf serum and 0.1% saponin (this antibody specifically colors the cell bodies and the neurites, allowing the study of neuronal cell death and the neural network).

This antibody will be revealed with an Alexa Fluor-conjugated secondary antibody at 1/400 dilution in PBS containing 1% FCS, 0.1% saponin, for 1 hour at room temperature. The cell nuclei will be counter-colored with a Hoechst solution (Sigma, 1/1000).

### Automatic Computer Analysis

For each condition, 30 images (representative of the entire area of the well) per well will be automatically taken using ImageXpress (Molecular Devices) with 20× magnification. All images are generated by ImageXpres® using the same acquisition parameters. From the images, the analyses will be directly and automatically carried out by MetaXpress® (Molecular Devices).

The following readings will be studied:
The total survival of the neurons (positive MAP-2 neurons, count),
The total network of neurites (MAP-2 in μm),

### Statistical Analysis

All the values are expressed in average +/−SEM (standard error of the mean). The statistical analysis will be carried out by one-way ANOVA, followed by a Dunnett or Fisher LSD test. p<0.05 will be considered significant.

### CONCLUSION

The results indicate that the composition according to the invention has neuroprotective effects of 0.3 μg/mL at 7.5 μg/mL (plate 1). DHA (40% extract) had a more modest neuroprotective effect, at 0.1 and 0.3 μM (plate 2).

The tested form of the composition according to the invention is the glycerinated form which comprises methyl cinnamate.

The results suggest that the glycerinated liquid form of the composition according to the invention could be a good candidate for joint pain since it is known in the literature that methyl cinnamate has demonstrated a potential anti-inflammatory activity with less cytotoxicity and good pro-inflammatory activity.

1. A composition comprising an extract derived from a mixture of plants consisting of Syzygium aromaticum, Santalum album, Aquilaria malaccensis, Boswellia carterii, Cyperus rotundus, Styrax benzoin, Liquidambar orientalis, Cinnamomum camphora, and Saussurea costus.

2. The composition according to claim 1, wherein the mixture of plants is an equal-mass mixture of plants.

3. The composition according to claim 1, characterized in that the extract is obtained from a mixture of plants which has:
a content of between 25 and 55% of Syzygium aromaticum,
a content of between 5 and 9% of Santalum album,
a content of between 15 and 30% of Aquilaria malaccensis,
a content of between 5 and 9% of Boswellia carterii,
a content of between 5 and 9% of Cyperus rotundus,
a content of between 5 and 9% of Styrax benzoin,
a content of between 5 and 9% of Liquidambar orientalis,
a content of between 5 and 9% of Cinnamomum camphora; and
a content of between 5 and 9% of Saussurea costus.

4. The composition of claim 1, characterized in that the extract is not a dry extract, preferably the extract is obtained by maceration in glycerin, more preferably the extract is obtained by maceration in vegetable glycerin.

5. The composition of claim 1, characterized in that the extract is derived from a mixture of Syzygium aromaticum buds, of Santalum album heartwood, of Aquilaria malaccensis heartwood, of Boswellia carterii resin, of Cyperus rotundus buds, of Styrax benzoin resin, of Liquidambar orientalis resin, of Cinnamomum camphora leaves, and of Saussurea costus roots.

6. The composition of claim 1, characterized in that it further comprises docosahexaenoic acid (DHA).

7. The composition of claim 1, characterized in that it is in the form of a gelcap, an oil or a functional beverage,

8. The composition of claim 1 to an of the preceding claims for use as a medication.

9. The composition of claim 1 to for use in the treatment and/or prevention of a neurodegenerative disease in mammals, preferably in humans.

10. The composition according to claim 9, characterized in that the neurodegenerative disease is Alzheimer's disease.
